(19) United States
(12) Patent Application Publication
(10) Pub. No.: US 2011/0144745 A1
(43) Pub. Date: Jun. 16, 2011

(54) OPHTHALMIC ENDOILLUMINATION SYSTEM

(76) Inventors: Michael McCulloch Martin, Newport Beach, CA (US); Michael James Papac, Tustin, CA (US); Andrew Mason Hahn, Anaheim, CA (US); Argelio Michael Olivera, Mission Viejo, CA (US)

(21) Appl. No.: 12/948,016

(22) Filed: Nov. 17, 2010

Related U.S. Application Data
(60) Provisional application No. 61/285,681, filed on Dec. 11, 2009.

(51) Int. Cl. A61F 2/14

(52) U.S. Cl. 623/4.1

(57) ABSTRACT

An endoillumination system comprises a system controller and a light modulation apparatus responsive to the controller to alter at least one parameter associated with a input light beam to generate a modulated light beam. The endoillumination system further comprises an optical fiber for carrying the modulated light beam and a probe through which at least a portion of the optical fiber extends. The probe is sized for insertion into an eye.

### RELATED APPLICATIONS

This application claims priority to U.S. provisional application Ser. No. 61/285,681, filed on Dec. 11, 2009, the contents which are incorporated herein by reference.

### TECHNICAL FIELD

This disclosure relates in general to an illuminator for use is ophthalmic surgery and more particularly to an ophthalmic illuminator using light modulation to adjust the qualities of the light output.

### BACKGROUND

Anatomically, the eye is divided into two distinct parts—the anterior segment and the posterior segment. The anterior segment includes the lens and extends from the outermost layer of the cornea (the corneal endothelium) to the posterior of the lens capsule. The aqueous humour fills the space between the lens and the cornea and helps maintain intraocular pressure. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of approximately 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. In a process known as vitreous syneresis, the collagen of the vitreous body may break down and result in retinal detachment.

Vitrectomy and other vitreoretinal surgical procedures are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

A surgeon performs vitreo-retinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

During such surgical procedures, proper illumination of the inside of the eye is important. Often, an endoilluminator, containing a small optical fiber is inserted into the eye to provide the illumination. A light source, such as a metal halide lamp, a halogen lamp, or a xenon lamp, is often used to produce the light carried by the optical fiber into the eye. Frequently the light output from the light source is inadequate and must be manipulated to achieve improved properties. For example, the intensity of the light beam may be attenuated using louvers in the optical path. However, louvers may have relatively large gratings and require motors or manual actuators for operation. Rotating louvers may also require significant space in the system. Correcting spatial nonuniformities of the propagating light beam is often performed using spatial filters. These spatial filters are an inconvenience because they are not adjustable and a series of filters may need to be swapped in and out to achieve the proper correction. This also introduces the potential for contaminants to enter the system. Adjusting the color of the light beam using color filters presents similar problems. Additionally, traditional methods of correcting chromatic aberration, such as colored ring formations, may be achieved using specially designed lenses. These corrective lenses are often expensive, difficult to manufacture, large in size, and specially engineered for a particular system.

New systems and methods are needed for adjusting the properties and quality of light used to provide illumination to the inside of the eye.

### SUMMARY

In one exemplary aspect, an endoillumination system comprises a system controller and a light modulation apparatus responsive to the controller to alter at least one parameter associated with an input light beam to generate a modulated light beam. The endoillumination system further comprises an optical fiber for carrying the modulated light beam and a probe through which at least a portion of the optical fiber extends. The probe is sized for insertion into an eye.

In another exemplary aspect, an ophthalmic endoillumination system comprises a light source for generating an input light beam and a light modulation apparatus comprising a pixel array for generating a modulated light beam. The system further comprises a controller in communication with the light modulation apparatus to alter at least one pixel in the pixel array and a condensing device for condensing the modulated light beam. The system further comprises an optical fiber for transmitting the condensed and modulated light beam and an endoillumination probe through which at least a portion of the optical fiber extends.

In another exemplary aspect, a method for ophthalmic endoillumination comprises providing a light source for producing an input light beam and modulating a light parameter with a light modulation apparatus to generate a modulated light beam. The method further comprises directing the modulated light bream onto an optical fiber and transmitting an output light beam from the optical fiber.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is a diagram of an endoillumination system according to one embodiment of the present disclosure.

FIGS. 2-7 depict various configurations of pixel arrays in the endoillumination systems of the present disclosure.

FIG. 8 is a cross section view of an endoillumination system positioned within an eye according to one embodiment of the present disclosure.

FIG. 9 is a flowchart describing a method of ophthalmic illumination.

FIG. 10 is a diagram of an endoillumination system according to another embodiment of the present disclosure.

FIG. 11 is a diagram of an endoillumination system according to another embodiment of the present disclosure.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

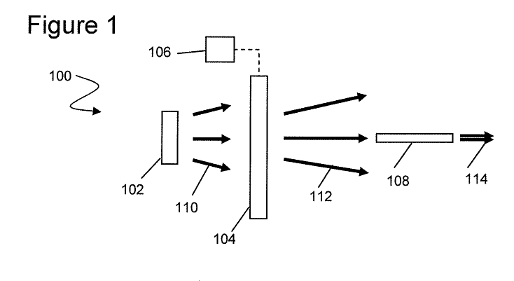

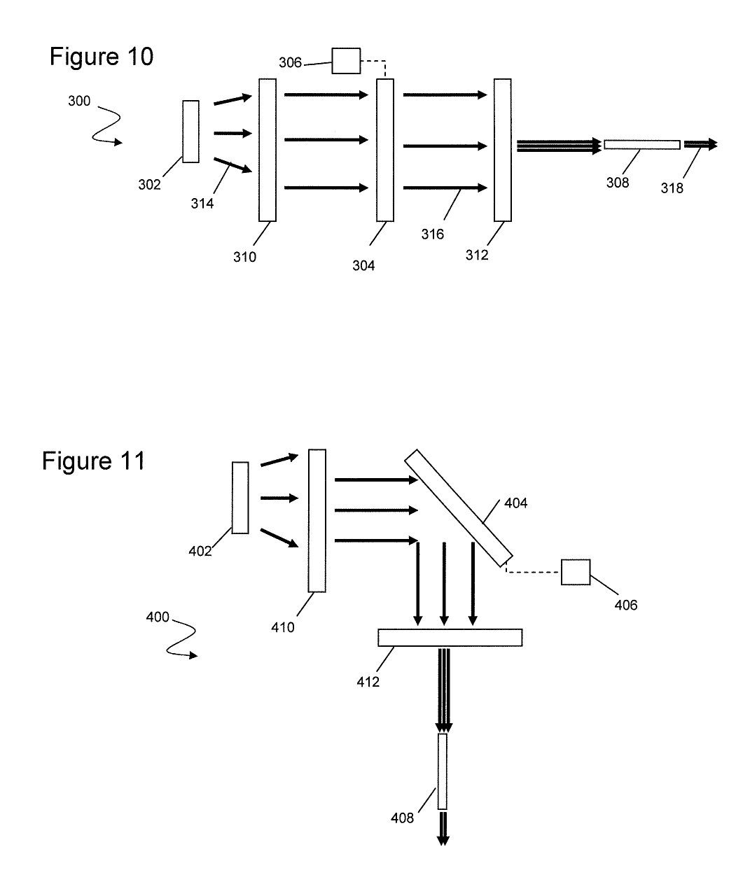

FIG. 1 shows an endoillumination system 100 including a light source 102, a light modulation apparatus 104, a controller 106, and an optical fiber 108. The light source 102 may include one or more light emitting diodes (LEDs), lasers, or a lamp including, for example, a metal halide lamp, a halogen lamp, a xenon lamp, a tungsten lamp, or any type of filament or gas lamp. The light source 102 generates a light beam 110. Although not shown in FIG. 1, the light beam 110 may be further adjusted through the use of filters, lenses, reflectors, prisms, or other structures to control the properties and direction of the light beam 110. See, e.g., FIGS. 10 and 11.

The light modulation apparatus 104 modulates the characteristics of the light beam 110 as will be described in detail. A light modulation apparatus may include a liquid crystal display (LCD), a liquid crystal on silicon (LCOS) device, a digital micro-mirror device (DMD), and/or other light modulation devices as would be apparent to one skilled in the art.

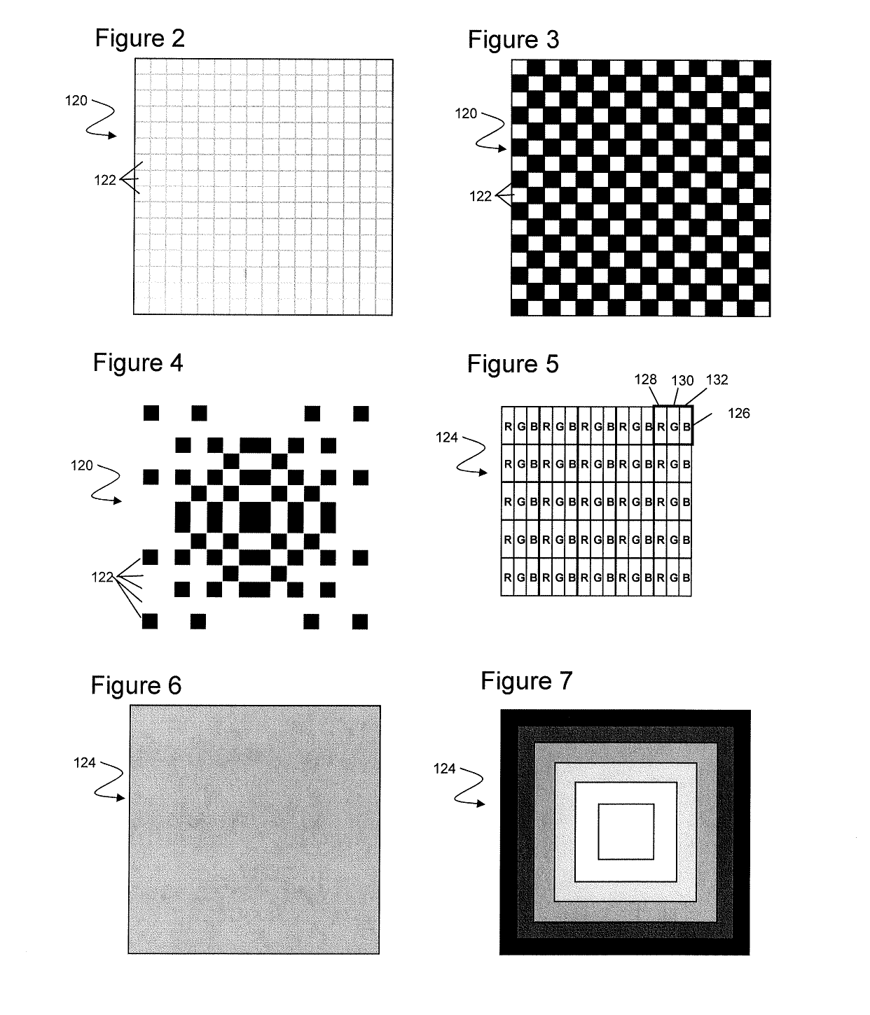

As shown in FIG. 1 and several of the exemplary embodiments of this disclosure, the light modulation apparatus 104 may be a liquid crystal apparatus such as an LCD. The LCD 104 is positioned in the optical path of the light beam 110. The LCD 104 in an electronically controlled panel comprising a pixel array 120 which includes a plurality of pixels 122 (See, e.g., FIGS. 2-4). Each pixel 122 comprises liquid crystal which can be manipulated to alter the parameters of the light beam 110 to generate a modulated light beam 112.

The controller 106, which may comprise hardware and software, provides control instructions to the LCD 104 based on any of a variety of input data including commands from a medical practitioner or other user, a predetermined program, or feedback received from a monitoring system which may detect characteristics of the input light beam 110, the modulated light beam 112, and/or an output light beam 114. The controller 106 may employ a variety of different control algorithms to control the LCD 104.

The controller 106 may communicate with the LCD 104 through a wired or wireless connection to control the pixel array 120. For example, the controller 106 may control the pixel array 120 to darken selected pixels to attenuate the light beam 110 and generate a modulated light beam 112 having a reduced intensity. In the example shown in FIG. 2, the pixel array 120 has no darkened pixels 122 which generates a modulated light beam 112 having substantially the same parameters as the input light beam 110. In the example shown in FIG. 3, the pixel array 120 has an evenly distributed number of darkened pixels 122 which comprise approximately fifty percent of the pixel array. This configuration of darkened pixels generates a modulated light beam 112 having approximately fifty percent light attenuation as compared to the input light beam 110.

As compared to traditional louver light attenuation devices, the system 100 may provide improved optical density in the modulated and output light. Bands of light and darkness generated by louvers may be reduced or eliminated. Additionally, space to rotate the louvers is no longer needed as the liquid crystal apparatus is stationary and relatively thin. Optically, the liquid crystal apparatus may be superior to traditional attenuation devices because the pixels are very small and are more evenly distributed than louvers. This finer resolution results in more uniformly distributed attenuation across an image.

In another example, the controller 106 may control the pixel array 120 to correct for spatial intensity nonuniformities in optical fiber output light beam 114. If monitoring of the output light beam 114, without any modulation of the light properties, indicates spatial nonuniformities such as a relatively bright center with a relatively dim periphery, the controller 106, may control the pixel array 120 to darken selected pixels to create a more uniform light intensity in the output beam 114. For example, as shown in FIG. 4, the pixels 122 near the center of the array 120 may be darkened with gradually fewer pixels darkened in the areas near the periphery of the array. This configuration of pixels may generate a modulated light beam 112 having a darker central region and a brighter peripheral region. As the modulated light beam 112 passes through the optical fiber 108, the fiber, as earlier noted, may tend to dim the brighter peripheral regions of the beam. The resultant output light beam may thus have more spatial light uniformity than an uncorrected light beam transmitted by the optical fiber 108.

As compared to traditional spatial filtering devices, such as neutral density filters, the system 100 may provide greater adjustability and thus may be more widely applicable. Because an LCD is controlled by software, the pattern on a single LCD can be manipulated to correct both stable and dynamic inconsistencies using feedback control. This allows for fine tuning of individual systems using non-specialized hardware. If the system characteristics drift over time, the same hardware can be used to correct a new configuration. Additionally, because the insertion and removal of filters is not required, the likelihood of contamination is reduced.

In another example, as shown in FIG. 5, the controller 106 may control a pixel array 124 to provide color correction or color filtering. It is understood that the pixel array 124 may also perform any of the light modulation described above for FIGS. 2-4. The pixel array comprises pixels 126, each of which include a set of subpixels 128, 130, and 132. In this example, each pixel 126 includes a red (R) subpixel 128, a green (G) subpixel 130, and a blue (B) subpixel 132. This is merely one example of a color pixel array and other configurations, pixel shapes, and quantities of subpixels per pixel may used in the alternative. The controller 106 may activate the color subpixels 128, 130, 132 in combination to generate a modulated light beam 112 having a selected color as shown in the pixel array of FIG. 6. Thus, the liquid crystal apparatus may serve as a color filter, allowing only light of specified wavelengths to pass through. A user may select different colors to accentuate particular illuminated features or may correct color properties of the light beam 110 to whiten or otherwise adjust the color of the modulated light beam 112.

As compared to traditional absorptive or dichroic filtering devices, the system 100 may provide greater color adjustability and thus may be more widely applicable. A single LCD may filter almost any color desired. By controlling the number of activated red, blue, and green pixels, almost any color in the visible spectrum may be projected. An LCD may be permanently contained within a system housing and requires no breeching of the encasement to change filter colors. Because the insertion and removal of filters is not required, the likelihood of contamination is reduced. Additionally, when the system housing is used as a Faraday cage to block external static electric fields, a system that does not require adding and removing filters may be preferable.

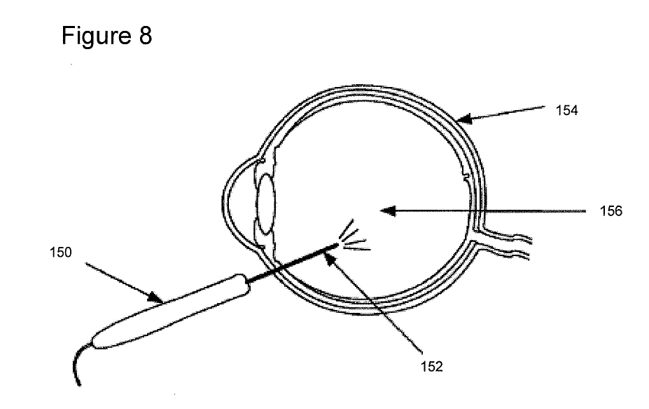

In another example, as shown in FIG. 7, the controller 106 may control the color pixel array 124 to offset chromatic aberration of the modulated light beam 112 or the output light beam 114 caused by other optical components in the system including optical fiber, lenses, or filters. In this example, the controller 106 may control the pixels and subpixels to create corrective patterns which offset the effect of the chromatic aberrations. For example, if the uncorrected output light beam 114 has a blue tint in the central region and a red tint in the peripheral regions, the pixel array 124 may be controlled to filter out more blue light in the central region and more red light in the peripheral regions. The resultant corrected output light beam 114 may be more spectrally uniform than the uncorrected beam. In addition to the corrective patterns and uniform color projections described above, the color pixel array 124 may also be used to project still or moving images by controlling the operation of the pixels and subpixels.

Traditional specialty lenses (e.g., achromat, trichromat, and hybrid refractive-diffractive lenses) used to correct chromatic aberrations are often custom made for a particular system. Because the system 100 may comprise more universally available and applicable components, the system 100 may provide a less expensive, easier to manufacture solution.

Many of the specific advantages of using a liquid crystal apparatus have been described above. As explained, the liquid crystal apparatus may perform the function of multiple other components. Combining these functionalities into a single liquid crystal apparatus conserves space and may reduce the cost of hardware and supporting electronics.

Referring now to FIG. 8, which depicts a hand piece 150 and a probe 152 in use. Probe 152 carries optical fiber 108 which may terminate in the eye to provide illumination. The optical fiber 108 may be a small gauge fiber. In certain embodiments, the optical fiber may be tapered. Hand piece 150 may include finger gripping surfaces or other ergonomic features which allow the user to maintain a comfortable grasp and manipulate the probe 152 within an eye. As shown, probe 152 may be inserted into an eye 154 through an incision in the pars plana region. Probe 152 illuminates the inside or vitreous region 156 of eye 154. In this configuration, the probe 152 may be used, for example, to provide illumination for vitreo-retinal surgery. Other insertion locations and surgical procedures that will benefit from the use of endoillumination will be clear to a person having ordinary skill in the art.

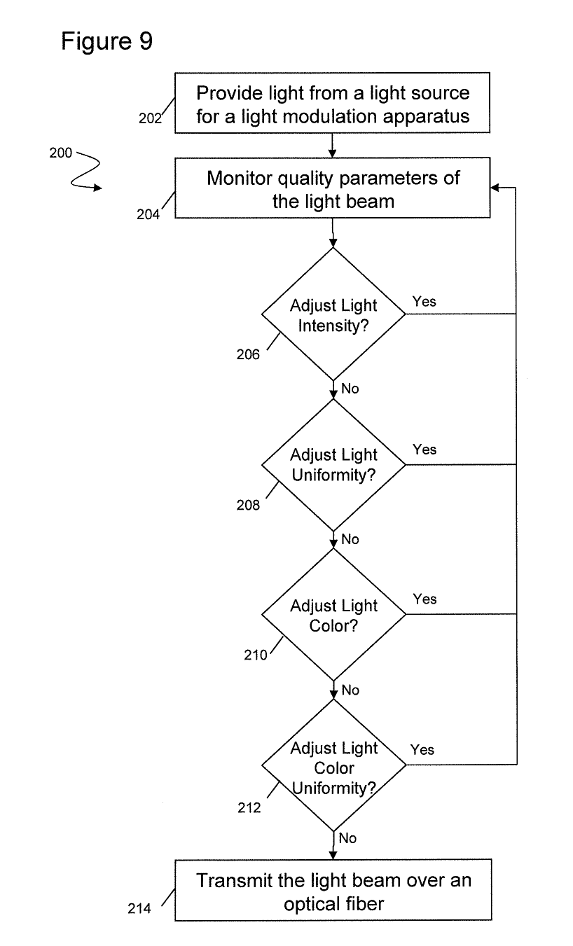

Referring now to FIG. 9, a method 200 for ophthalmic illumination using endoillumination system 100 is provided. At step 202, the light source 102 provides light for the light modulation apparatus such as the liquid crystal apparatus 104. At step 204, quality parameters such as light intensity, uniformity of light intensity, color, and chromatic uniformity may be monitored anywhere along the optical path, including prior to the liquid crystal apparatus 104, after the liquid crystal apparatus, or after transmission through the optical fiber 108. At step 206, a user or a computer associated with the controller 106 may determine whether and how much to adjust the intensity of the monitored light. Based upon this determination, the liquid crystal apparatus 104, under the control of controller 106, may selectively adjust the brightness of the pixels in the pixel array. At step 208, a user or computer associated with the controller 106 may detect spatial nonuniformities and determine corrective action. Based upon this determination, the liquid crystal apparatus 104, under the control of controller 106, may selectively darken pixels in the pixel array to correct for the spatial nonuniformities. At step 210, a user or computer associated with controller 106 may detect light color and determine whether it should be altered. Based upon this determination, the liquid crystal apparatus 104, under the control of the controller 106, may selectively change the color or all or a portion of the pixels in the pixel array to adjust the light color. At step 212, a user or computer associated with controller 106 may detect light color nonuniformities and determine corrective action. Based upon this determination, the liquid crystal apparatus 104, under the control of controller 106, may adjust select pixels in the pixel array 200 to create more uniform light color. After each step 206-212, the modulated light 112 or the output light 114 may be further monitored and further adjustment or correction may be made. At step 214, the modulated light 112 may be transmitted along the optical fiber 108 to illuminate, for example, the interior tissues of an eye. The method 200 may be performed while the probe 150 carrying the optical fiber 108 is inserted into an eye or may be performed while the probe 150 is entirely external of the eye, such as during preparation for surgery.

Referring now to FIG. 10, in an alternative embodiment, an endoillumination system 300 may comprise a light source 302, a light modulation apparatus 304, a controller 306, and an optical fiber 308. These components of the system 300 may be identical or substantially similar in structure and/or function to the components of the same name identified in endoillumination system 100. The system 300 may further comprise a collimating lens 310 positioned between the light source 302 and the light modulation apparatus 304, which is a liquid crystal apparatus in this embodiment. The collimating lens 310 may be a fiber lens, ball lens, aspherical lens, graded index (GRIN) lens, cylindrical lens, or any other type of device, including optical fiber, which can be used to collimate light into a substantially parallel light beam. The system 300 may further comprise a condensing lens 312 which may be, for example, a biconvex or plano-convex spherical lens, an aspheric lens, or any other type of device which can be used to focus a light beam for launching the beam into a small diameter optical fiber. In this embodiment, an input light beam 314 from light source 302 may be collimated by collimating lens 310 before passing to the liquid crystal apparatus 304. A modulated light beam 316 generated by liquid crystal apparatus 304 may be focused, using condensing lens 312, to pass through the optical fiber 308. An output light beam 318 is transmitted from the optical fiber 308.

In this embodiment, the liquid crystal apparatus 304 may be controlled by the controller 306 in the same or similar manner as previously described for the liquid crystal apparatus 104 to adjust the uniformity, intensity, color, or color patterns in the modulated light beam 316. In alternative embodiments, the collimating lens 310 and the condensing lens 312 may be positioned on the same side, (either the input or output side) of the liquid crystal apparatus 304 or the lenses 310 may be connected or integrally formed. It is further understood that additional lenses, mirrors, or other optical devices may be used to further control the direction and properties of the light passing through endoillumination system 300.

Referring now to FIG. 11, in this embodiment, an endoillumination system 400 may comprise a light source 402, a light modulation apparatus 404, a controller 406, and an optical fiber 408. The system 400 may further comprise a collimating lens 410 positioned between the light source 402 and the light modulation apparatus 404 and a condensing lens 412 positioned between the light modulation apparatus 404 and the optical fiber 408. In this embodiment, the light modulation apparatus 404 is reflective rather than transmissive. It may controlled by the controller 406 to perform the same light modulation functions as described above. A suitable reflective light modulation apparatus may be a liquid crystal on silicon (LCOS) device. It us understood that additional reflective surfaces, such as mirrors, DMDs, lenses, prisms, or other optical devices may be used to control the direction and properties of the light passing through endoillumination system 400.

Although several selected embodiments have been illustrated and described in detail, it will be understood that they are exemplary, and that a variety of substitutions and alterations are possible without departing from the spirit and scope of the present invention, as defined by the following claims.

We claim:

1. An endoillumination system comprising:
a system controller;
a light modulation apparatus responsive to the controller to alter at least one parameter associated with a input light beam to generate a modulated light beam;
an optical fiber for carrying the modulated light beam; and
a probe through which at least a portion of the optical fiber extends, the probe sized for insertion into an eye.

2. The endoillumination system of claim 1
wherein the light modulation apparatus comprises a liquid crystal apparatus.

3. The endoillumination system of claim 1
wherein the light modulation apparatus comprises an array of pixels adjustable in response to the controller.

4. The endoillumination system of claim 3
wherein the brightness of each pixel in the array of pixels is selectively adjustable in response to the controller.

5. The endoillumination system of claim 3 wherein the array of pixels comprise a plurality of pixel sets wherein each pixel set is selectively adjustable in response to the controller.

6. The endoillumination system of claim 5 wherein each pixel set comprises a red pixel, a blue pixel, and a green pixel.

7. The endoillumination system of claim 1 further comprising at least one collimating lens.

8. The endoillumination system of claim 1 further comprising at least one condensing lens.

9. The endoillumination system of claim 1 wherein the light modulation apparatus transmits at least a portion of the input light therethrough to generate the modulated light beam.

10. The endoillumination system of claim 1 wherein the light modulation apparatus reflects at least a portion of the input light to generate the modulated light beam.

11. An ophthalmic endoillumination system comprising:
a light source for generating an input light beam;
a light modulation apparatus comprising a pixel array for generating a modulated light beam;
a controller in communication with the light modulation apparatus to alter at least one pixel in the pixel array;
a condensing device for condensing the modulated light beam;
an optical fiber for transmitting the condensed and modulated light beam; and
an endoillumination probe through which at least a portion of the optical fiber extends.

12. The ophthalmic endoillumination system of claim 11 wherein the light modulation apparatus comprises a liquid crystal apparatus.

13. The ophthalmic endoillumination system of claim 11 wherein the pixel array comprises a plurality of pixel sets responsive to the controller to modulate the color of the input light beam.

14. The ophthalmic endoillumination system of claim 10 wherein the pixel array is configured to transmit at least a portion of the input light beam to generate the modulated light beam.

15. The ophthalmic endoillumination system of claim 10 wherein the pixel array is configured to reflect at least a portion of the input light beam to generate the modulated light beam.

16. A method for ophthalmic endoillumination comprising:
providing a light source for producing an input light beam;
modulating a light parameter with a light modulation apparatus to generate a modulated light beam;
directing the modulated light bream onto an optical fiber; and
transmitting an output light beam from the optical fiber.

17. The method of claim 16 further including inserting at least a portion of the optical fiber into an eye.

18. The method of claim 16 wherein the light modulation apparatus is a liquid crystal display.

19. The method of claim 16 wherein the light modulation apparatus is a liquid crystal on silicon panel.

20. The method of claim 16 wherein the light parameter is light intensity.

21. The method of claim 16 wherein the light parameter is spatial uniformity.

22. The method of claim 16 wherein the light parameter is color.

23. The method of claim 16 wherein the light parameter is color uniformity.

24. The method of claim 16 wherein the optical fiber is a single optical fiber.

25. The method of claim 16 further comprising monitoring the input light beam and responsive to the monitoring, providing a control signal to the light modulation apparatus to modulate the light beam.

26. The method of claim 16 further comprising monitoring the modulated light beam and responsive to the monitoring, providing a control signal to the light modulation apparatus to modulate the light beam.

27. The method of claim 16 further comprising monitoring the output light beam and responsive to the monitoring, providing a control signal to the light modulation apparatus to modulate the light beam.
